(19) United States
(12) Patent Application Publication
(10) Pub. No.: US 2021/0106803 A1
(43) Pub. Date: Apr. 15, 2021

(54) SUBCUTANEOUS INFUSION CANNULAS HAVING A PLURALITY OF APERTURES FOR DOSAGE DISTRIBUTION OVER A WIDE AREA OF SUBCUTANEOUS TISSUE

(71) Applicant: Victor Kaiser-Pendergrast, Stockholm, NJ (US)

(72) Inventor: Victor Kaiser-Pendergrast, Stockholm, NJ (US)

(21) Appl. No.: 16/598,723

(22) Filed: Oct. 10, 2019

(51) Int. Cl. A61M 39/02; A61M 25/00

(52) U.S. Cl. CPC A61M 39/0247; A61M 25/007; A61M 25/0097; A61M 2039/0282; A61M 25/0102; A61M 2210/04; A61M 2039/0205; A61M 2039/0291; A61M 2039/0276

(57) ABSTRACT

A cannula tubing having a series of apertures at its distal end, so that the end of the tube is not the only egress point for the payload of the cannula. The cannula tubing is affixed to a base, which is configured to accept an input connector on the end opposing the cannula. The base is configured so that input medication from the connector flows down the cannula. The base is affixed to the patient via adhesive. The perforated cannula distributes the dose of the medication across a wider area. This has been demonstrated to increase uptake rate of some medications (reducing the time to peak absorption rate). Administering medication (such as insulin) over a greater area may lessen unwanted side effects of subcutaneous injecting, such as lipohypertrophy, scarring, or localized drug resistance.

### FIELD OF THE INVENTION

The present invention generally relates to subcutaneous infusion cannulas.

### BACKGROUND OF THE INVENTION

Traditional subcutaneous infusion cannulas (for example, those used with insulin pumps) are simply tubes made of steel, Teflon®, or other bio-compatible material, that are used to administer medication for absorption in the subcutaneous tissue. Traditional cannulas deposit their payload into a concentrated area at the tip of the cannula, and may be held manually for a short duration, or alternatively fastened to the skin's surface via adhesive.

FIGS. 1A-1C illustrates a traditional subcutaneous infusion cannula 001. The distal end 010 of the cannula 001 is inserted under the skin. Traditional cannulas are inserted either perpendicularly into the skin, or at a shallow angle. This example cannula tube would be inserted under the skin about halfway (or more) of the total length of the cannula. The length of a typical infusion cannula is under 20 mm. In this example, the proximal end of the cannula 001 is attached to a hub 040. The hub 040 has a flange 030 with an adhesive on its bottom surface, configured to attach to a patient's skin. Hub 040 is configured with a connector 050 on its end opposing cannula 001. The connector 050 is configured to mate with male connector and accept an input tube that supplies medication to the hub 040. The medication is supplied through the cannula 001, depositing the medication from the distal end 010 of the cannula into the patient's subcutaneous tissue.

There are other cannulas with various aperture patterns in the sidewall, but none designed expressly for the purpose of infusing medication over a multi-day period. For instance, US20140163323 A1 (Laparoscopic cannula with suturing capability) shows a cannula with sidewall holes intended for the passage of needles, designed for use in laparoscopic surgery. U.S. Pat. No. 8,771,199 B2 (Full core biopsy needle with secondary cutting cannula) is used in surgical environments. WO2014074606 A1 (Adjustable liposuction cannula) is composed of inner and outer members, with the outer sheath having holes in it; this is used in liposuction procedures, not the infusion of medication.

WO2002058607 A2 (Infusion cannula) with “a pair of diametrically opposed delivery holes”, used in vitreoretinal surgeries to deliver an infusate to maintain fluid pressure in the eye during a surgery. The use and operation of this type of cannula is quite different from continuous delivery of a medication subcutaneously, as the infusate is used only to maintain fluid pressure, and the cannula is only used for a short period during a surgery.

US 20040236313 A1 (Infiltration Cannula) describes a cannula with a plurality of apertures arranged around the cannula tube's distal end. This infiltration cannula features a hub configured to be held manually during a surgical procedure.

U.S. Pat. No. 4,723,947 A describes an insulin compatible infusion cannula configured to connect to an external medication source.

US 20140074033 A1 describes an insulin infusion set, configured to be attached to the patient's skin via adhesive. This device differs from the device and method of the present invention, in that the device of the present invention includes a plurality of apertures in the cannula tubing.

As well as many other surgical cannula patents, not related to subcutaneous infusion of medication, improving medication uptake rate, or minimizing possible skin and subcutaneous side effects resulting from prolonged infusion.

### SUMMARY OF THE INVENTION

A cannula according to one embodiment of the present invention has a plurality of apertures formed along the sidewall of the tubing of the cannula so that the end of the tube is not the only egress point for the payload of the cannula. The cannula is attached to a hub on one end. The hub has an adhesive affixed to its bottom, configured to attach to a patient's skin. The hub has a connector on its end, opposing said cannula tubing. The connector is configured to accept an input tube that supplies medication to the hub and through to the cannula. The perforated cannula of the present invention distributes the dose of the medication across a wider area than traditional cannulas. This has been demonstrated to increase the uptake rate of some medications (reducing the time to peak absorption rate). Administering medication (such as insulin) over a greater area lessens unwanted local side effects of subcutaneous injection, such as lipohypertrophy, scarring, or localized drug resistance.

The apertures at only the tip of the cannula of a cannula as described in WO2002058607 do not accomplish the objective of the present novel subcutaneous infusion cannula described herein. A goal of the present invention is to spread delivery of the medication along the length of the cannula, to maximize medication uptake speed and minimize potential local side effects of infusion.

The infiltration cannula described in US 2004/0236313 differs from the device and method of the present invention, in that the present invention securely fastens the infusion cannula to the patient's skin by adhesive for continuous infusion over longer periods of time. The purpose of the plurality of apertures in the infiltration cannula of US 2004/0236313 is to distribute the payload for the purpose increasing the area covered by a localized effect. The present invention differs in that the present invention (being attached by adhesive) distributes a continuously delivered payload over a larger area for the purpose of reducing localized tissue damage over time, as well as increasing the rate of the subcutaneous tissue's absorption of the payload.

The infusion cannula described in U.S. Pat. No. 4,723,947 differs from the device and method of the present invention, in that the present invention includes apertures in the cannula tubing sidewall and a base configured to attach to the patient's skin via adhesive.

A subcutaneous infusion cannula according to an embodiment of the present invention includes a biocompatible tubing having a sidewall, a proximal end and a distal end, wherein said distal end is configured to be inserted subcutaneously. The sidewall of the tubing has a plurality of apertures formed therein. The tubing further has an interior diameter that is sufficient to accept a rigid insertion needle therethrough to puncture the patient's skin and allow subcutaneous insertion of the tubing. The cannula further has a hub that is configured to be affixed by adhesive to the skin of the patient receiving treatment. The hub has a distal end attached to the proximal end of the biocompatible tubing and a proximal end configured to accept an input tubing. The distal end of the hub is configured to receive a fluid from the input tubing, the fluid flowing through the hub and through the biocompatible tubing and out the plurality of apertures formed in the sidewall of the biocompatible tubing.

In a method according to one embodiment of the present invention, a medical fluid is provided into a patient's subcutaneous tissue. The method includes using a rigid insertion stylet to insert an infusion cannula's biocompatible tubing through a patient's skin into the subcutaneous tissue. The infusion cannula includes a biocompatible tubing having a sidewall, a proximal end and a distal end. The biocompatible tubing has a sufficient internal diameter to accept said rigid insertion stylet; and a plurality of apertures in the sidewall. The infusion cannula further includes a hub coupled to the biocompatible tubing. The hub has an input connector for accepting an input tubing. The method further involves affixing the hub to the patient using an adhesive and removing the rigid stylet from the biocompatible tubing, leaving the biocompatible tubing positioned in the patient's subcutaneous tissue. The input tubing is connected to the hub's input connector and fluid is supplied from the input tubing, through the hub, and into the biocompatible tubing inserted into the patient's subcutaneous tissue. According to the claimed method finally ejects the input fluid from the biocompatible tubing into the patient's subcutaneous tissue via the apertures.

A subcutaneous infusion cannula according to another embodiment of the present invention includes two biocompatible tubings. Each of the tubes has a sidewall, a proximal end and a distal end. The distal ends of the tubes are configured to be inserted subcutaneously. Each of the biocompatible tubings further has an interior diameter sufficient to accept a rigid insertion needle therein. The cannula also includes a hub configured to be affixed by adhesive to a skin surface of a patient receiving treatment. The hub has a distal end having outlets attached to the proximal ends of the two biocompatible tubings. The hub further has a proximal end having an inlet configured to accept a single input tubing. The hub also has an internal channel communicatively coupled to the inlet and the outlets. A fluid received from the input tubing flows through the internal channel to the biocompatible tubings and out the distal ends of the biocompatible tubing.

### BRIEF DESCRIPTION OF THE DRAWINGS

For the purposes of illustrating the present invention, there is shown in the drawings a form which is presently preferred, it being understood however, that the invention is not limited to the precise form shown by the drawing in which:

FIGS. 1A-1C depict a traditional subcutaneous infusion cannula with adhesive, typically used with continuous infusion insulin pumps;

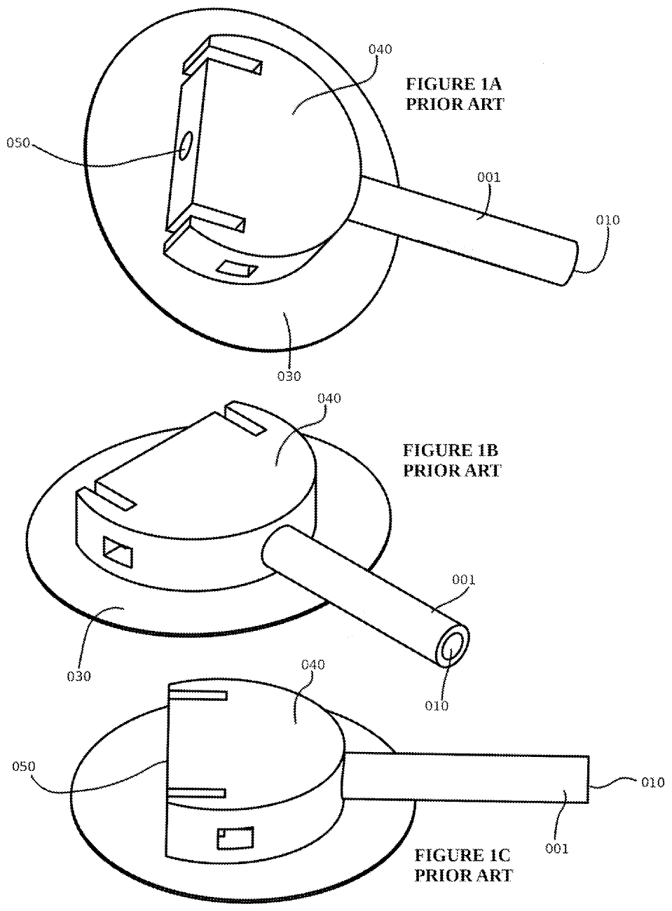

FIGS. 2A and 2B illustrate an infusion cannula with a slot cut in the tubing's sidewall through the end of the tubing;

FIGS. 3A-3C depict an infusion cannula with a slot cut in the sidewall that is not cut through the end of the tubing;

FIGS. 4A-4C illustrate an infusion cannula with multiple apertures in the sidewall;

FIGS. 5A-5C is an infusion cannula with multiple apertures of different sizes cut in the tubing's sidewall;

FIGS. 6A-6C depict an infusion cannula with apertures in the sidewall at varying angle (spiral) which delivers the payload to varying depths of subcutaneous tissue;

FIGS. 7A-7C illustrate an infusion cannula with slots in the bottom of sidewall which delivers the payload into subcutaneous tissue underneath the cannula;

FIGS. 8A-8C depict a cannula with slits in the tubing's bottom, and further depicts a tapered tip, intended to equalize flow of medication;

FIG. 9 depicts a traditional rigid insertion stylet device capable of running down the length of the tubing of the device depicted in FIGS. 1A-1C;

FIGS. 10A-10D depict the device in FIG. 3, shown with the insertion stylet device from FIG. 9;

FIGS. 11A and 11B are of an infusion set with two cannula tubings, each of which is perforated with slits cut along the bottom;

FIG. 12 is a double rigid insertion stylet designed for use with an infusion set with two cannula tubings;

FIGS. 13A and 13B are an infusion set with two cannula tubings with plain sidewalls;

FIGS. 14A-14C depict the infusion set from FIG. 13, along with the double insertion stylet from FIG. 12;

FIGS. 15A and 15B depict an input connector designed to be used with dual cannula tubing device of FIGS. 11A, 11B, 13A, and 13B;

FIGS. 15C and 15D depict a cutaway view of the device of FIGS. 15A and 15B illustrating that the single input tubing splits to two output tubes;

FIG. 16 is a flowchart of the method for using the perforated subcutaneous infusion cannula; and

FIG. 17 illustrates the device of FIG. 4 as it may be inserted in a patient, with the cannula tubing inserted under the skin, such that the tip and apertures broadly deposit the payload medication into the patient's subcutaneous tissue.

### DETAILED DESCRIPTION OF THE INVENTION

Subcutaneous cannulas are often associated with scarring or other side effects as a result of injecting medication at the same location for an extended period of time. For insulin, a concern is lipohypertrophy at the infusion site, caused by repeatedly injecting insulin at the same injection site (see Robert Young, James Hannan, Brian Frier, Judith Steel and Leslie Duncan, Diabetic Lipohypertrophy Delays Insulin Absorption, Diabetes Care, 7(5):479-480 (September-October 1984). More significantly, lipohypertrophy has been demonstrated to slow insulin absorption (see id). By using a perforated cannula (or an infusion set with multiple cannula tubings), the medication is infused over a larger volume of subcutaneous tissue, and thus the concentration of medication at any one point is lessor than it would be with the traditional cannula, wherein all medication egresses through the end of the tube, into a small localized site. This has the potential of reducing unwanted side effects of cannula infusion, such as lipohypertrophy, and avoiding delayed insulin uptake caused by the lipohypertrophy.

Another concern regarding multi-day use of traditional subcutaneous cannulas is occlusion of the cannula, and the ensuing failure to deliver medication. With more egress points, a cannula with perforations is less likely to occlude completely, thus avoiding a critical failure of traditional subcutaneous cannulas. A survey of pediatric patients using subcutaneous insulin infusion found that 71% of patients had problematic events with their cannulas, 33.9% of those events being occlusion in the cannula, and therefore failed doses (see Lutz Heinemann And Lars Krinelke, Insulin Infusion Set: The Achilles Heel Of Continuous Subcutaneous Insulin Infusion, Journal of Diabetes Science and Technology, 6:954-964 (July 2012)).

Heinemann summarizes a study on cannula wear, stating that 30% of cannulas are removed after an event of “hyperglycemia following a failed correction dose,” implying that either the infusion site area has become drug resistant or the cannula has occluded (see Lutz Heinemann, Insulin Infusion Sets: A Critical Reappraisal, Diabetes Technology & Therapeutics, 18(5):327-333 (November 2016)). The perforated cannulas of the present invention solve both of these major unaddressed problems (see id.). Furthermore, Heinemann summarizes another study showing that on just the third day of wear, a cannula delivering insulin loses efficacy, as mean blood glucose is higher in patients using cannulas that have been inserted more than two days (see id.). Heinemann also finds that in this study, the mean fasting blood glucose increases for every day that a cannula is inserted. Assuming that this is a result of localized drug resistance (and not trauma, given that the cannula has been in use for two days) (see id.), the perforated cannulas of the present invention alleviate this issue by distributing the dose across a wide area of tissue, instead of bombarding a highly localized area of subcutaneous tissue with the entirety of the cannula's payload. This may extend beyond insulin usage to other drugs administered with a subcutaneous cannula.

Heinemann's survey also references a study showing that 42% of younger Type I diabetic patients using insulin pumps had lipohypertrophy, a percentage consistent with other surveys and studies (see id.). Lipohypertrophy is a frequent occurrence known to be specifically caused by continued injection of insulin in the same site. The cannulas of the present invention, by spreading insulin (or other medication) doses across the subcutaneous tissue, skin conditions like lipohypertrophy may be reduced.

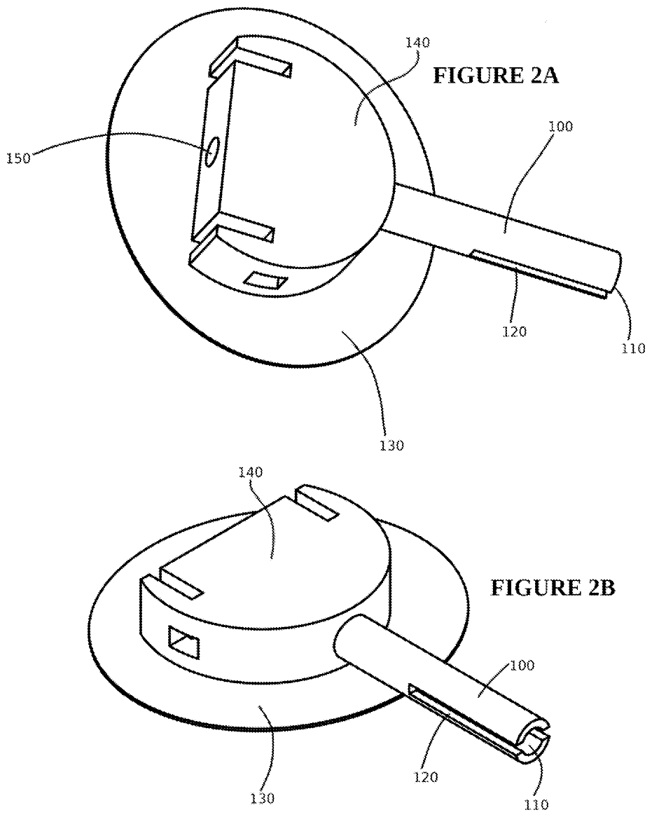

FIGS. 2A and 2B illustrates an embodiment of the present invention. The infusion cannula 100 shown in FIG. 2 has diametrically opposed slots, or slits, 120 cut into the sidewall of the cannula 100 tubing, so that the cannula payload is delivered out the diametrically opposed slots 120 and distal end 110 of the cannula tubing 100. As shown in these Figures, the slits 120 extend in a longitudinal direction down the cannula tubing 100. The cannula tubing 100 is attached to the cannula hub 140. An adhesive 130 patch on the bottom of the cannula hub 140 allows for temporary attachment to the patient's skin. The end of the cannula hub 140 opposite cannula 100 is configured with a connector 150 to accept an input source of the cannula's payload. In this embodiment, the slots 120 extend all the way through the distal end 110 of the cannula 100 and the distal end 110 is open. A typical cannula 100 tubing may be 10 mm to 20 mm in length. The exterior diameter of a typical cannula 100 tubing may be between 0.5 mm and 1.0 mm, while the wall of the cannula 100 tubing may be 0.05 to 0.25 mm thick. The slots 120 may be roughly half of the length of the cannula 100, perhaps 5 mm to 10 mm in length. The width of the slots 120 may be in the range of 0.05 mm to 0.3 mm, such that sufficient rigidity of the cannula 100 tubing is retained. For most medications that are delivered subcutaneously, it is important that the payload is delivered into the subcutaneous fat. For patients with minimal subcutaneous fat, it is necessary to ensure that any payload medication is not delivered below the thin layer of adipose tissue. By forming the diametrically opposed slots 120 in parallel with the skin's surface, the slots will be delivering payload into subcutaneous adipose tissue, instead of any underlying muscle or other tissue.

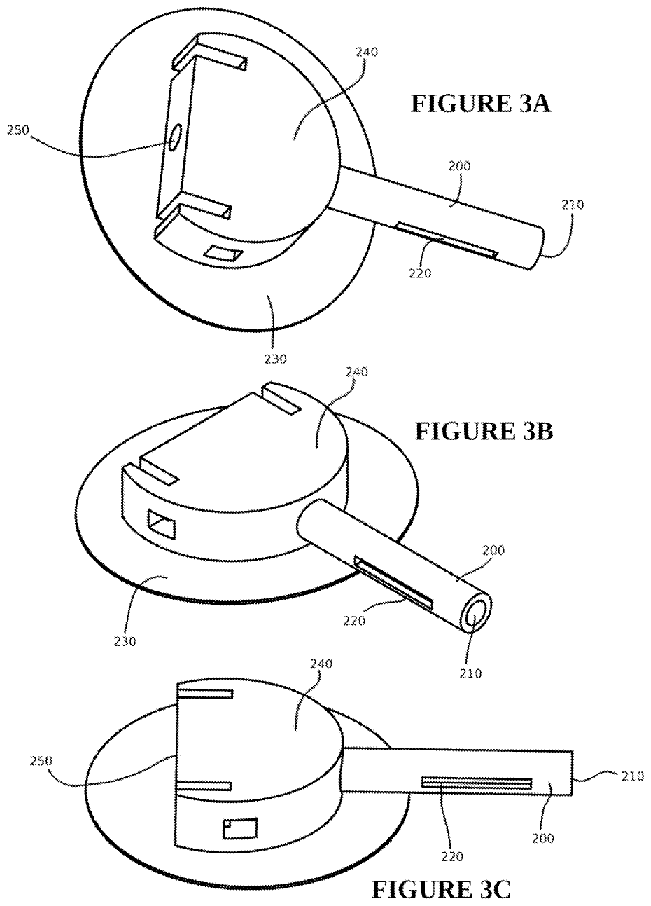

FIGS. 3A-3C demonstrate a cannula 200 with diametrically opposed slots 220 cut into the sidewall of the cannula 200 tubing. This embodiment is similar to the one illustrated in FIGS. 2A-2B, except the slots 220 do not extend all the way to the end of the cannula tubing. Forming slots that do not extend to the end of the cannula 200 preserves the cannula 200 tubing's rigidity. The cannula 200 is attached to the cannula hub 240. An adhesive patch 230 is affixed to the bottom of the cannula hub 240 to facilitate attachment of the hub 240 to the patient's skin. The end of the cannula hub 240 opposite cannula 200 is configured with a connector 250 to accept an input source of the cannula's payload. The slots 220 formed may be roughly one third the length of the cannula tubing, making the length of the slots 220 between 6 mm and 12 mm.

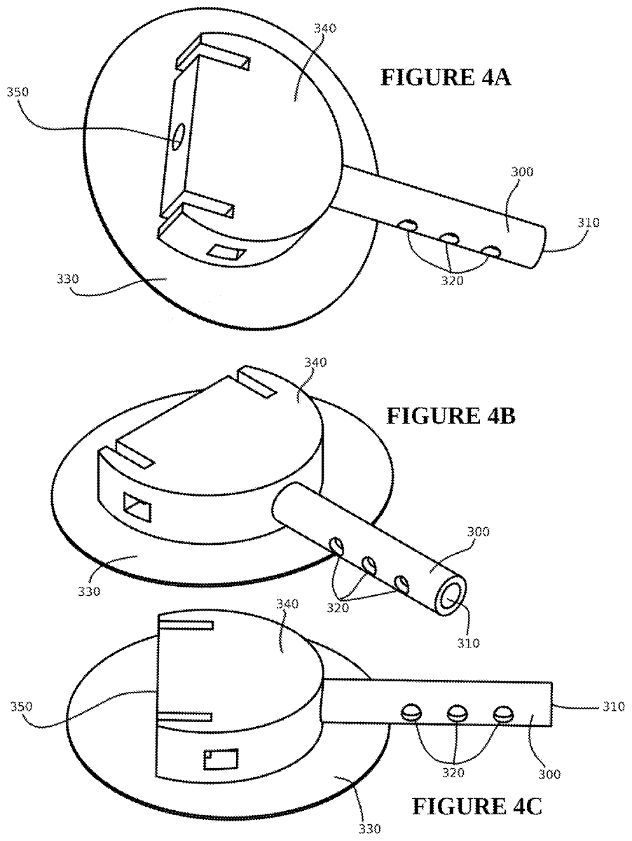

A preferred embodiment of the present invention is shown in FIGS. 4A-4C. In this embodiment, apertures, 320 are formed in the sidewall of the cannula 300, so that cannula payload is delivered not only through the distal end 310 of the cannula tubing, but also out the side holes 320. Subcutaneous infusion cannulas are often under one millimeter in diameter, and so complex modifications of the tubing can be difficult. Punching, drilling, or otherwise creating holes in the sidewall of the cannula, through both walls of the tubing is practical to machine and spreads the cannula's payload over a wide area of subcutaneous tissue. The cannula 300 is attached to the cannula hub 340. The end of the cannula hub 340 opposite cannula 300 is configured with a connector 350 to accept an input source of the cannula's payload. The apertures 320 may have diameter between 0.05 mm and 0.3 mm and may be spaced 2 mm to 5 mm apart. Since the length of the typical cannula 300 may be between 10 mm to 20 mm, this embodiment may have one to eight apertures 320 formed in the sidewall of the cannula 300. The preferred embodiment displayed in FIGS. 4A-4C has three apertures 320 formed.

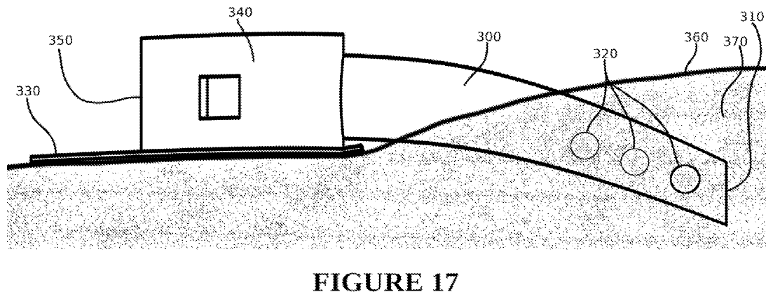

In FIG. 17, the device of FIGS. 4A-4C is shown with the cannula tubing 300 inserted through the patient's skin 360 and into the patient's subcutaneous tissue 370. The apertures 320 in the sidewall of the cannula tubing and the cannula's distal end 310 are in the patient's subcutaneous tissue 370, and the adhesive 330 affixed to the cannula hub 340 is attached to the patient's skin 360, holding the infusion cannula in place during delivery of the payload.

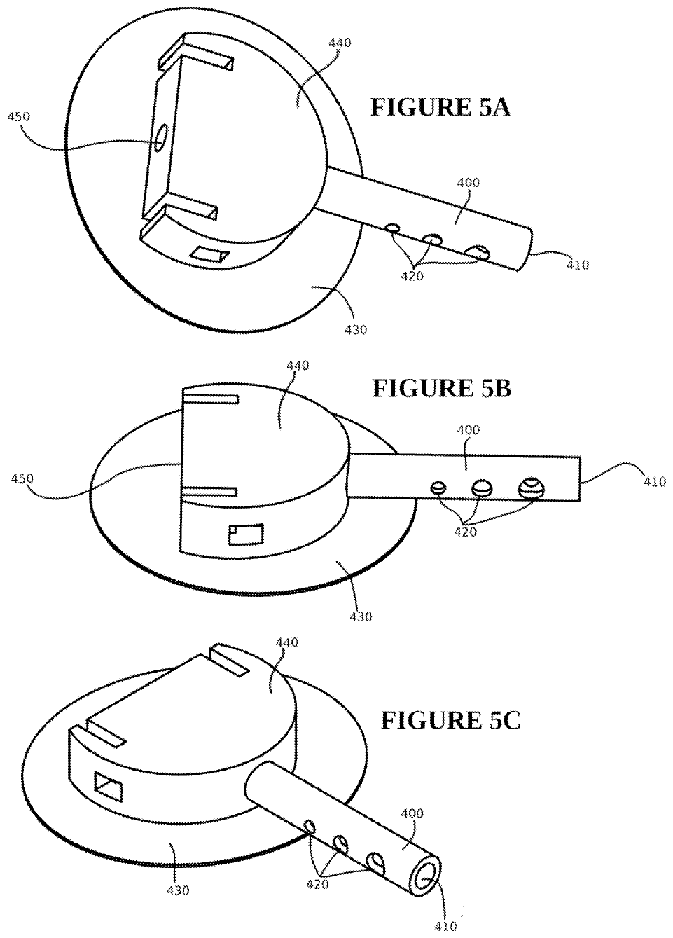

The embodiment displayed in FIGS. 5A-5C has diametrically opposed holes 420 of varying sizes manufactured in the sidewall of the cannula tubing 400. By manufacturing holes of different sizes, the payload of the cannula may be distributed equally through the holes 420 down the length of the cannula tubing, as well as the distal end 410 of the cannula. The largest holes may have diameter equal to that of the cannulas distal end, perhaps 0.6 mm in diameter. The smallest holes may be only a fraction of that diameter, perhaps 0.1 mm in diameter. The cannula 400 is attached to the cannula hub 440. The end of the cannula hub 440 opposite cannula 400 is configured with a connector 450 to accept an input source of the cannula's payload.

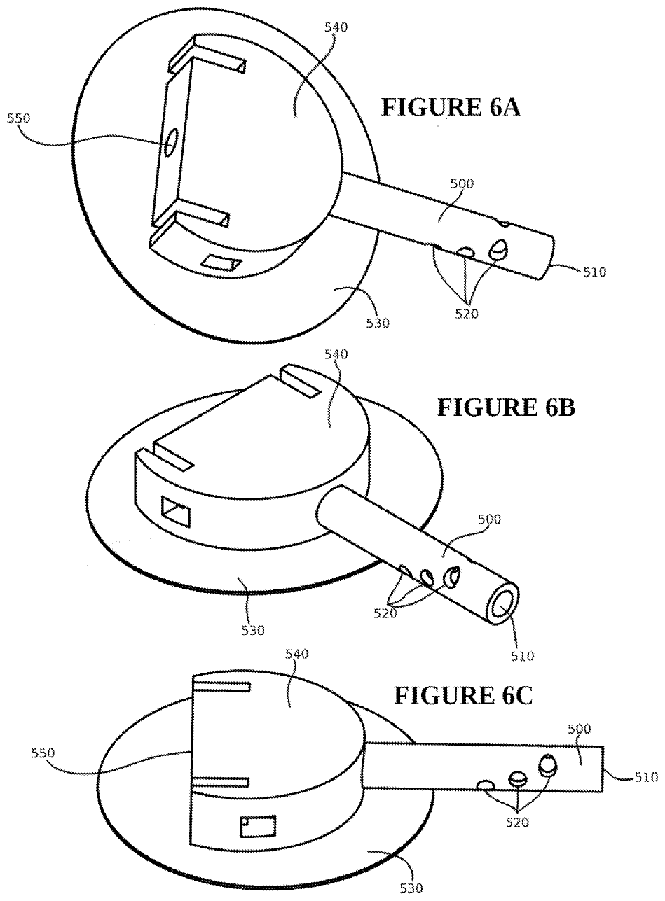

FIGS. 6A-6C illustrate an infusion cannula where a non-linear pattern of diametrically opposed apertures 520 have been manufactured in the sidewall of the cannula tubing 500. In this embodiment, the holes 520 spiral around the cannula tube. By manufacturing holes in a non-linear pattern, the payload of the cannula may be broadly distributed through the holes 520 and the distal end of the cannula 510 into the patient's subcutaneous tissue. The cannula 500 is attached to the cannula hub 540. The end of the cannula hub 540 is configured with a connector 550 to accept an input source of the cannula's payload.

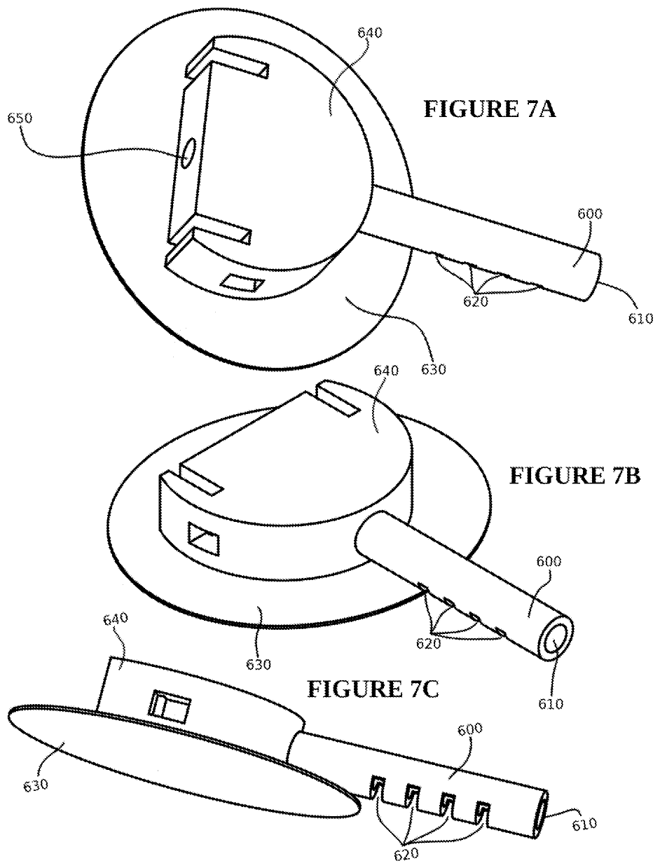

FIGS. 7A-7C display an embodiment of the present invention, where the infusion cannula 600 has circumferential slots 620 cut into the bottom of the cannula tubing. As shown in these Figures, the slots 620 extend transverse to an axial direction of the biocompatible tubing 600. By forming slots cut in the bottom of the cannula tubing, the cannula's payload may be distributed further from the skin of the patient through the slots 620 and the distal end of the cannula 610. By “bottom” of the cannula 600, what is meant is the ventral portion of tube facing away from the patient's skin, rather than the dorsal portion of the tube 600 that faces the skin. The cannula 600 is attached to the cannula hub 640. The end of the cannula hub 640 is configured with a connector 650 to accept an input source of the cannula's payload. As used herein, biocompatible indicates material whose presence in biological tissue does not lead to harmful local or systemic effects in the host. Biocompatible materials are resistant to corrosion, as corrosion would lead to negative local or systemic effects. Examples of biocompatible materials include, for example, surgical-grade stainless steel, titanium, fluoropolymers, polyvinylchloride, polyurethane, and many other plastics.

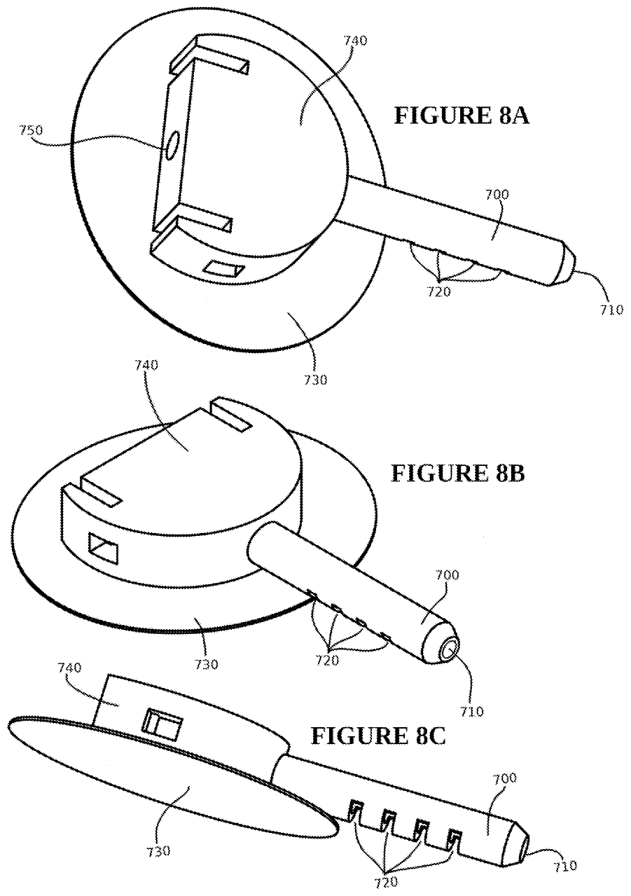

The infusion cannula illustrated in FIGS. 8A-8C has slots 720 cut into the bottom of the cannula tubing 700, as well as a tapered distal end 710. Tapering the tip reduces payload flow from the distal end 710 of the cannula, this can alter the payload's flow from the slots 720 and the distal end 710. Reducing the flow from the distal end 710 may be beneficial by distributing the payload more evenly among the slots 720 and the distal end 710. The cannula 700 is attached to the cannula hub 740. the end of the cannula hub 740 is configured with a connector 750 to accept an input source of the cannula's payload. The design of the tapered distal end 710 may be additionally applied on any of the other embodiments.

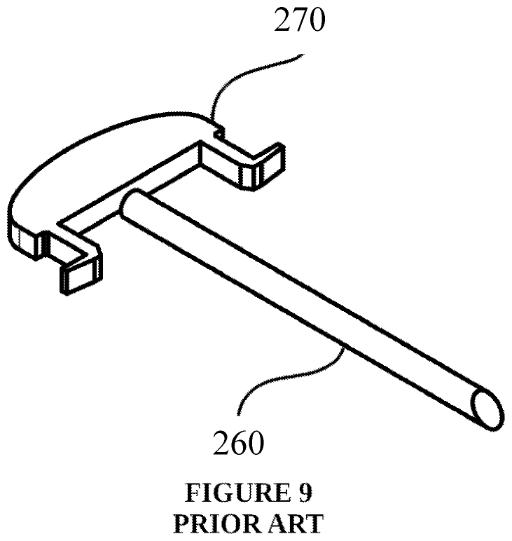

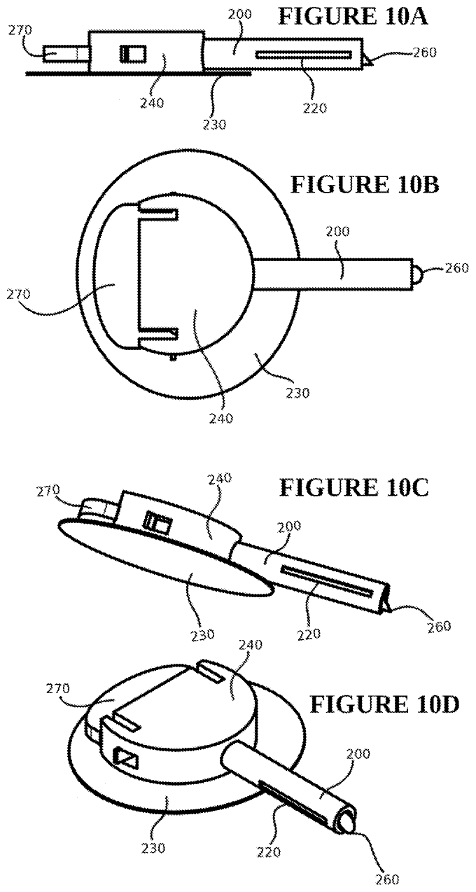

FIGS. 10A-D illustrates the device of FIGS. 3A-3C with the insertion stylet 260 of FIG. 9 running down the length of the cannula tubing 200. The insertion stylet connector 270 is connected to the cannula hub 240, keeping the insertion stylet in place during insertion. The cannula tubing has an interior diameter sufficiently large to accept an insertion stylet/needle 260 therethrough. When the patient inserts a new cannula, the insertion stylet 260 first pierces the patient's skin. As the patient continues to guide the cannula tubing into the skin, the rigid insertion stylet 260 ensures the cannula tubing 200 remains straight and rigid under the skin. After the cannula tubing 200 and insertion stylet 260 are fully inserted, the patient affixes the adhesive patch 230 to the skin, keeping the cannula tubing in position under the skin. Finally, the patient disconnects the insertion stylet connector 270 and pulls the insertion stylet assembly 260 and 270 completely out from the cannula tubing 200 and cannula hub 240. The patient may now connect an input source to the cannula hub 240, and begin dosing payload through the cannula tubing 200, whereby the payload is distributed to the patient's subcutaneous tissue from both the slots 220 and distal end of the cannula 200. The same insertion stylet assembly may be used to insert the other similar embodiments with a single cannula tubing.

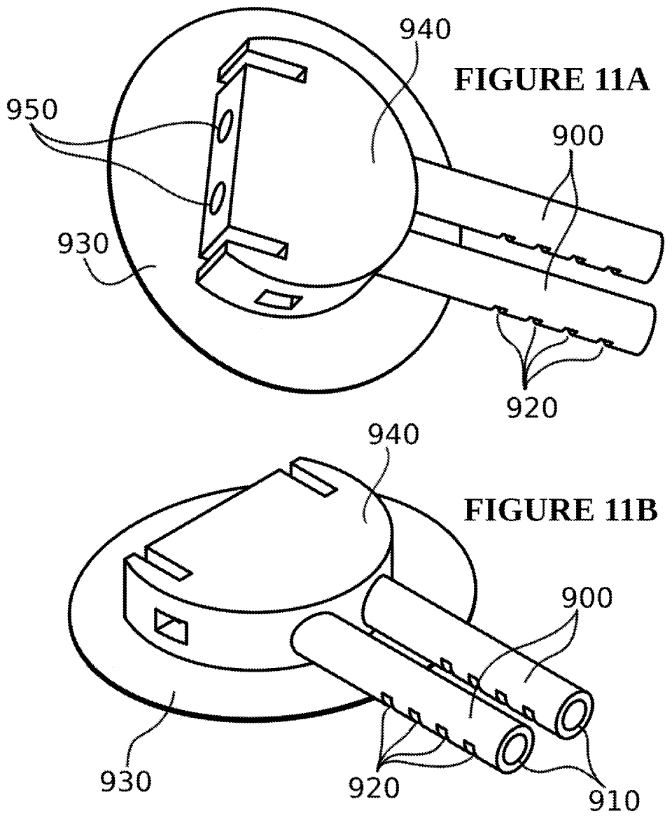

The device illustrated in FIGS. 11A and 11B has two cannula tubings 900 on a single cannula hub 940. Both cannula tubings 900 have slots 920 formed in their bottoms. By having multiple cannula tubing 900s, the payload of the infusion cannula is spread over an even wider area of subcutaneous tissue. The end of the cannula hub 940 is configured with a female connector 950 to accept an input source of the cannula's payload. The payload is transmitted through the connector 950, through the cannula hub 940, into both of the cannula tubings 900, and is output through the slots 920 and distal ends 910 of the cannula tubings.

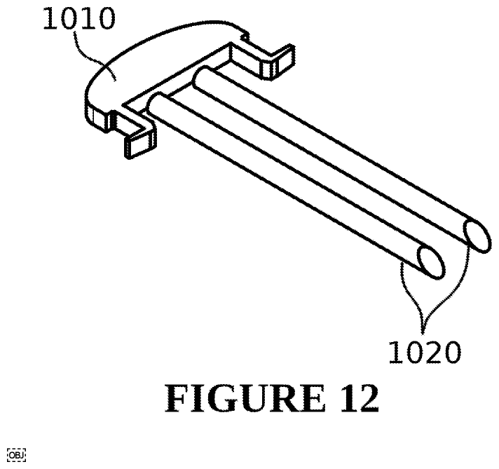

FIG. 12 displays insertion stylets 1020 that may be used with an infusion cannula that has two cannula tubings. The insertion stylets are affixed to a connector 1010 that may be connected to a cannula hub 940 (See FIGS. 11A, 11B). This stylet may be used in a similar manner as the stylet displayed in FIG. 9, but for infusion cannulas that incorporate two cannula tubings.

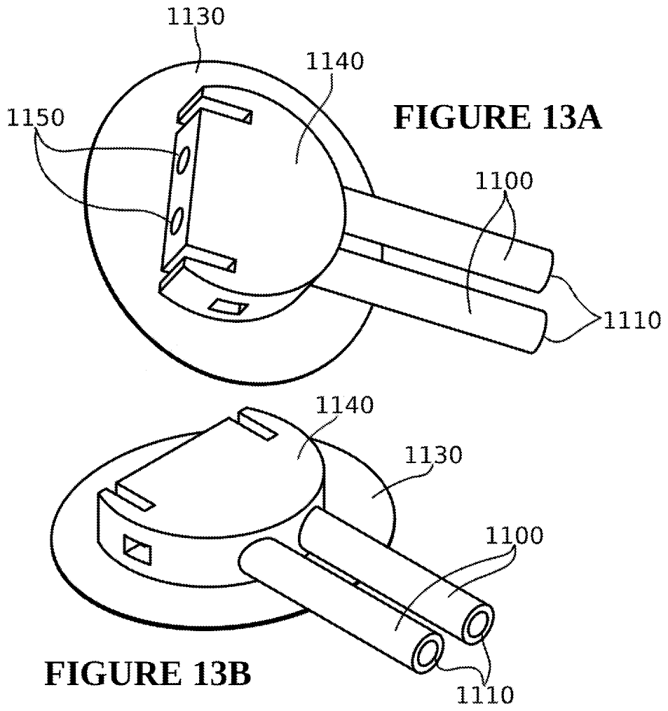

FIGS. 13A and 13B display an infusion cannula with two cannula tubings 1100 affixed to a single cannula hub 1140. The cannula tubings 1100 do not have sidewall perforations, but by having two cannula tubings, the payload is still delivered over a wider area of subcutaneous tissue than a traditional cannula delivers payload to. The end of the cannula hub 1140 opposite the cannulas 1100 is configured with a connector 1150 to accept an input source of the cannula's payload. The payload is transmitted through the connector 1150, through the cannula hub 1140, into both of the cannula tubings 1100, and out the distal ends 1110 of the cannula tubings 1100 into the patient's subcutaneous tissue. As appreciated by those skilled in the art, other embodiments of the infusion cannula with two cannula tubings may incorporate sidewall aperture designs shown in FIGS. 2A-2B, 3A-3C, 4A-4C, 5A-5C, 6A-6C, and/or the tapered tip design illustrated in FIGS. 8A-8C.

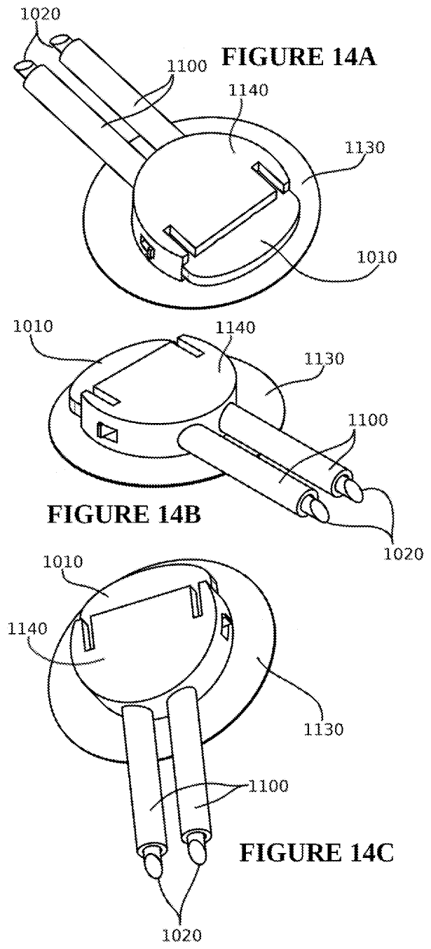

FIGS. 14A-14C illustrates the device of FIGS. 13A and 13B with the insertion stylets 1020 from FIG. 12 inserted through both cannula tubings 1100. The insertion stylet connector 1010 is connected to the cannula hub 1140.

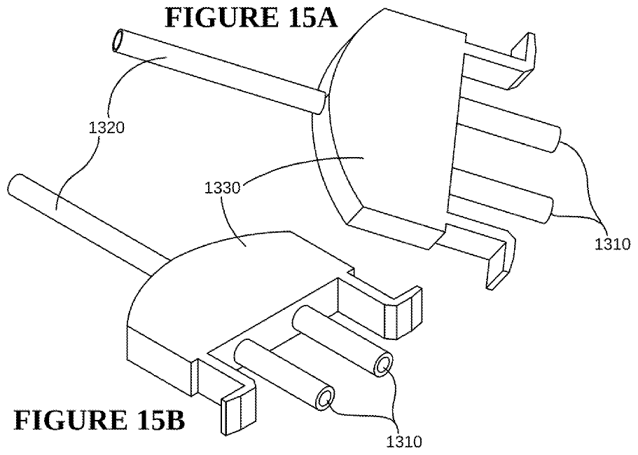

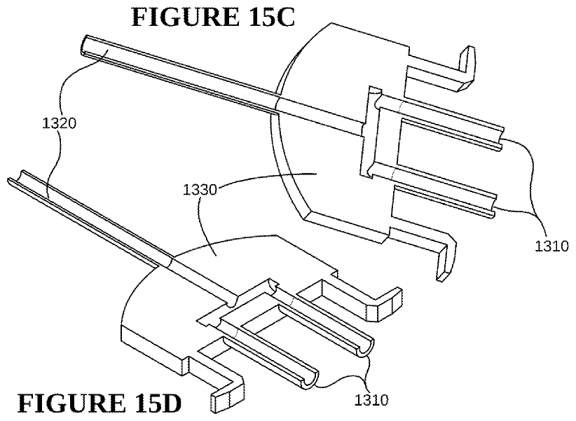

FIGS. 15A and 15B display an input, male connector for use with an infusion cannula that has two cannula tubings. A flexible input tubing 1320 receives the payload from an input source, piping it into a connector base 1330.

The connector base has an internal channel that splits the payload from an inlet, the input tubing 1320, to two outlets, output tubes 1310, that connect with the input connectors of the hub and infusion cannula that has two cannula tubings and two input connectors. FIGS. 15C and 15D depict a cutaway view of the same device shown in FIGS. 15A and 15B, where the top halves of the connector base 1330, input tubing 1320, and output tubes 1310 are removed for illustrative purposes. This cutaway view depicts the interior channel formed in connector base 1330 that transmits the incoming payload from the single flexible input tubing 1320 to both output tubes 1310.

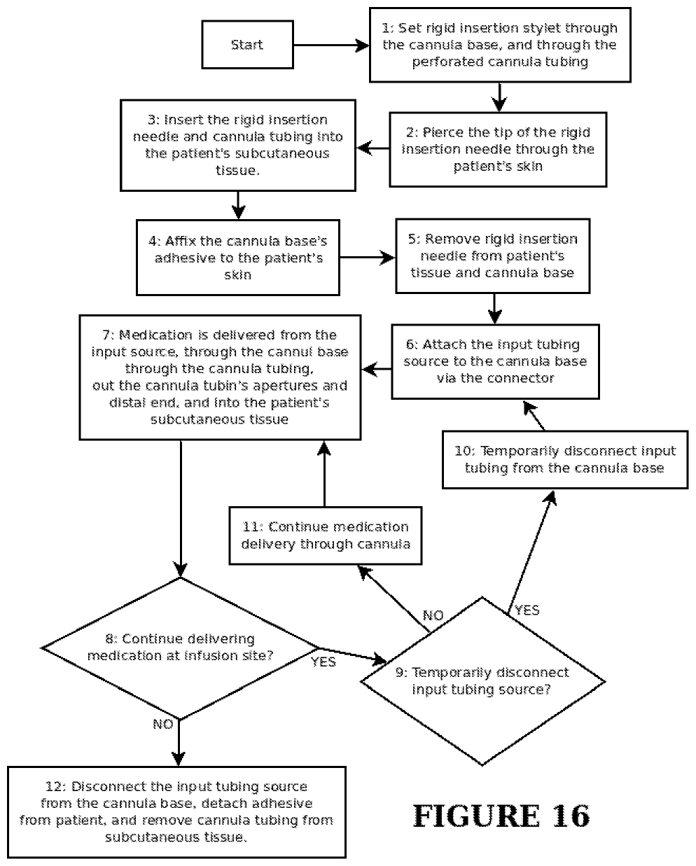

FIG. 16 describes the method of using the subcutaneous infusion cannula for delivering payload to a patient's subcutaneous tissue. Step 1: the rigid insertion stylet is run through the cannula base and through the cannula tubing. The tip of the insertion stylet may be extended beyond the distal end of the cannula tubing. Step 2: the tip of the insertion stylet is used to pierce the patient's skin, creating a small puncture that may be used for inserting the cannula tubing. Step 3: the insertion stylet and cannula tubing are pushed through the opening in the patient's skin, until the cannula tubing is fully positioned in the patient's subcutaneous tissue. The cannula tubing must be inserted far enough such that the apertures are under the skin, in the subcutaneous tissue. Step 4: the adhesive patch on the cannula base is affixed to the patient's skin. This ensures the cannula is securely positioned under the patient's skin and allows for delivery of the payload for extended time frames. Step 5: now that the cannula is under the patient's skin and the adhesive is securing the cannula's position under the skin, the rigid insertion style is no longer required. The insertion stylet is pulled out of the subcutaneous tissue and cannula tubing. This frees the input connector for acceptance of an input source. Step 6: an input tubing source is attached to the cannula base. Step 7: Now that the input source is attached, medication is delivered from the input source. The payload medication is transmitted through the cannula base and through the cannula tubing, before being delivered out of the cannula tubing apertures and distal end, into the subcutaneous tissue. Medication may be delivered on a continuous basis, or as discretionary boluses. Step 8: After medication has been delivered, it may be desirable to discontinue usage of the medication with this cannula tubing. There are many reasons why a patient or care provider may wish to discontinue usage of the infusion site. In the case of personal continuous infusion insulin pumps, the FDA has approved other infusion cannulas for three days of usage, after which period the infusion cannula must be replaced at a new infusion site; regular replacement of cannulas is generally considered to be a good practice to preemptively avoid infections. Other reasons to discontinue usage of the infusion site include the development of redness, swelling, or irritation at the infusion site. It may also be desirable to replace infusion cannulas more frequently to avoid localized scarring that occurs from persistent usage. Finally, the patient's or caregiver's own discretion may be used to decide when to change the cannula tubing, based on factors related to the patient's health, personal preferences, lifestyle, or any other factor. Step 9: It may be necessary to switch input sources or temporarily disconnect the input source. For example, if the input source is an electronic continuous infusion insulin pump, it may be required that the patient disconnect said input insulin pump before bathing Step 11: If it is not necessary to disconnect the input source, then continue dosing payload medication as normal. Step 10: If the input tubing must be temporarily disconnected from the cannula base, then proceed to do so. Later, an input tubing will be reconnected, and delivery of the medication will be resumed, in Step 6. Step 12: Once it is determined that medication delivery is completed at this cannula's insertion site, the input tubing is disconnected, the adhesive patch is unfixed from the patient's skin, and the cannula tubing is pulled out from the subcutaneous tissue. At this point, it may be desirable for the patient to insert a new cannula elsewhere on the patient's body.

Some or all of the sidewall perforations may be covered in a biodegradable material. In this embodiment, these perforations may “open up” sometime after being inserted subcutaneously, as the biodegradable material dissolves in the body. This allows the subcutaneous tissue receiving the dose to change over time, as new egress points become available as the biodegradable material dissolves.

Other embodiments of a subcutaneous cannula with sidewall perforations are possible.

Other embodiments of a subcutaneous cannula with multiple cannula tubings with sidewall perforations are possible.

Although the present invention has been described in relation to particular embodiments thereof, many other variations and other uses will be apparent to those skilled in the art. It is preferred, therefore, that the present invention be limited not by the specific disclosure herein, but only by the gist and scope of the disclosure.

What is claimed is:

1. A subcutaneous infusion cannula comprising:
a biocompatible tubing having a sidewall, a proximal end and a distal end, wherein said distal end is configured to be inserted subcutaneously;
the sidewall having a plurality of apertures formed therein;
the biocompatible tubing having an interior diameter sufficient to accept a rigid insertion needle therethrough; and
a hub configured to be affixed by adhesive to a skin surface of a patient receiving treatment, the hub having a distal end attached to the proximal end of the biocompatible tubing and a proximal end configured to accept an input tubing, the proximal end of the hub configured to receive a fluid from the input tubing, the fluid flowing through the hub and through the biocompatible tubing and out the plurality of apertures formed in the sidewall of the biocompatible tubing

2. The cannula of claim 1, wherein the plurality of apertures comprises substantially circular holes in the biocompatible tubing.

3. The cannula of claim 2, wherein the plurality of apertures extends linearly along the sidewall between the distal and proximal ends of the biocompatible tubing.

4. The cannula of claim 2, wherein the plurality of apertures extends in a spiral pattern along the sidewall between the distal and proximal ends of the biocompatible tubing.

5. The cannula of claim 2, wherein the plurality of apertures varies in size between the distal and proximal ends of the biocompatible tubing.

6. The cannula of claim 5, wherein the plurality of apertures increases in size between the distal and proximal ends of the biocompatible tubing.

7. The cannula of claim 2, wherein the plurality of apertures comprise at least one pair of substantially circular apertures formed in diametrically opposed portions of the sidewall.

8. The cannula of claim 1, wherein the plurality of apertures comprise slits formed in the sidewall.

9. The cannula of claim 8, wherein the slits extend longitudinally through the distal end of the biocompatible tubing.

10. The cannula of claim 8, wherein the slits are formed in diametrically opposed portions of the sidewall.

11. The cannula of claim 8, wherein the slits are formed in the sidewall transverse to an axial direction of the biocompatible tubing.

12. The cannula of claim 11, wherein the biocompatible tubing has a dorsal portion facing the skin and a ventral portion facing the subcutaneous tissue when inserted subcutaneously, wherein the slits are formed in the ventral portion of the biocompatible tubing.

13. The cannula of claim 1, wherein a tip of the biocompatible tubing at the distal end is tapered.

14. The cannula of claim 1, further comprising a biodegradable material covering at least one of the apertures, wherein as the biodegradable material dissolves, a size of the at least one aperture increases.

15. The cannula of claim 1, wherein the biocompatible tubing is a biodegradable material

16. A method of providing medical fluids into a patient's subcutaneous tissue, the method comprising:
using a rigid insertion stylet to insert an infusion cannula's biocompatible tubing through a patient's skin into the subcutaneous tissue, the infusion cannula comprising:
a biocompatible tubing having a sidewall, a proximal end and a distal end, wherein the biocompatible tubing has sufficient internal diameter to accept said rigid insertion stylet; and a plurality of apertures in the sidewall; and
a hub coupled to the biocompatible tubing, the hub having an input connector for accepting an input tubing;
affixing the hub to the patient using an adhesive;
removing the rigid stylet from the biocompatible tubing, leaving the biocompatible tubing positioned in the patient's subcutaneous tissue;
connecting the input tubing to the hub's input connector;
transporting fluid from the input tubing, through the hub, and into the biocompatible tubing inserted into the patient's subcutaneous tissue; and
ejecting the input fluid from the biocompatible tubing into the patient's subcutaneous tissue via the apertures.

17. The method in claim 16 where the medical fluid is insulin or an insulin analogue.

18. A subcutaneous infusion cannula comprising:
two biocompatible tubings, each tubing having a sidewall, a proximal end and a distal end, wherein the distal ends are configured to be inserted subcutaneously; and
each of the biocompatible tubings having an interior diameter sufficient to accept a rigid insertion needle therein; and
a hub configured to be affixed by adhesive to a skin surface of a patient receiving treatment, the hub having:
a distal end having outlets attached to the proximal ends of the biocompatible tubings;
a proximal end having an inlet configured to accept a single input tubing,
an internal channel communicatively coupled to the inlet and the outlets whereby a fluid received from the input tubing flows through the internal channel to the biocompatible tubings and out the distal ends of the biocompatible tubing.

19. The device of claim 18 wherein a sidewall of at least one of the biocompatible tubings has a plurality of apertures.

20. The device of claim 18 wherein the internal channel of the hub is provided in a detachable connector.
